(19) United States
(12) Patent Application Publication
(10) Pub. No.: US 2007/0161960 A1
(43) Pub. Date: Jul. 12, 2007

(54) Lancet device

(75) Inventors: Yen-Hui Chen, Taipei (TW); Fu-Yuan Li, Wantan Hsiang (TW); Tzer-Ming Chen, Taipei (TW)

Correspondence Address: PATENTTM.US, P. O. BOX 82788, PORTLAND, OR, 97282-0788, US

(73) Assignees: Fu-Yuan Li; Yen-Hui Chen

(21) Appl. No.: 11/331,522

(22) Filed: Jan. 12, 2006

(51) Int. Cl. A61M 5/00

(52) U.S. Cl. 604/187

(57) ABSTRACT

A lancet device has a needle assembly and a needle carrier. The needle assembly has a casing, a needle hub, a retracting unit and a limit unit. The needle hub with a needle is mounted in the casing. The retracting unit and the limit unit connect to the needle hub to limit the needle hub to move. The lancet carrier has a housing, a pushing rod, a spring and a cap. The housing connects the needle assembly and has a pressing unit. The pushing rod is mounted in the housing and abuts with the needle hub after pushing the pressing unit to release the force of the spring so the needle protrudes from the needle assembly. After pressing the cap, the needle assembly releases from the needle carrier. Therefore, the new needle assembly can be mounted in the lancet carrier to prick again.

### BACKGROUND OF THE INVENTION

1. Field of the Invention

The present invention relates to a lancet device, and especially to a lancet device for obtaining small amount blood conveniently.

2. Description of the Related Art

A conventional lancet device comprises a casing and a needle hub. The casing comprises a connector and a driver assembly. The connector and the driver assembly are mounted in the casing. The needle hub is mounted in the casing and comprises a needle. The needle hub is connected with the casing by the connector. The driver assembly is attached on the needle hub. When pushing the driver assembly, the needle is protruded from the needle hub and pricks into the skin of a person. Then, the needle is retracted into the needle hub to suck some blood into the casing through the needle. The driver assembly dose not return to the original position until the used needle hub is replaced with a new needle hub. However, a used needle hub maybe reused again due to a user's carelessness, so another person may be infected by the used needle hub.

### SUMMARY OF THE INVENTION

The objective of the present invention is to provide a lancet device that can be used a needle once to protect a next person who is pricked his skin by the used needle.

To achieve the foregoing objective, a lancet device having a needle assembly and a needle carrier is provided. The needle assembly has a casing, a needle hub, a retracting unit and a limit unit. The casing is cylindrical and hollow and has an inner surface, a rear stop, a front stop and a neck. The neck is formed on the casing near the front end, The rear stop and the front stop are formed on the inner surface in sequence. The needle hub with a needle is mounted in the casing. The retracting unit connects the needle hub, abuts with the inner surface and corresponds to the neck. The limit unit connects the needle hub and abuts with the rear stop when the needle hub is at an original position. The lancet carrier has a housing, a pushing rod, a spring and a cap. The housing connects the needle assembly and has a pressing unit. The pushing rod is mounted in the housing and abuts with the needle hub after pushing the pressing unit to release the force of the spring so the needle protrudes from the needle assembly. After pressing the cap, the needle assembly releases from the needle carrier. Therefore, the new needle assembly can be mounted in the lancet carrier to prick again.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a lancet device in accordance with the present invention;

FIG. 2 is an exploded perspective view of a needle assembly of the lancet device in the FIG. 1;

FIG. 3 is a side view in partial section of the needle assembly in the FIG. 2;

FIG. 4 is a front view of the needle assembly in the FIG. 2;

FIG. 5 is an exploded perspective view of a lancet carrier of the lancet 1 device in the FIG. 1;

FIG. 6 is a side view in partial section of the lancet device in the FIG. 1;

FIG. 7 is a side view in partial section of the needle assembly in the FIG. 2 showing a needle protruding from the needle assembly;

FIG. 8 is an operational side view in partial section of the needle assembly in the FIG. 2 showing the needle retracting in the needle assembly; and

FIG. 9 is an exploded side view in partial section of the lancet device in the FIG. 1 showing the used needle assembly being released from the lancet carrier.

### DETAILED DESCRIPTION OF THE PREFERRED EMBODIMENTS

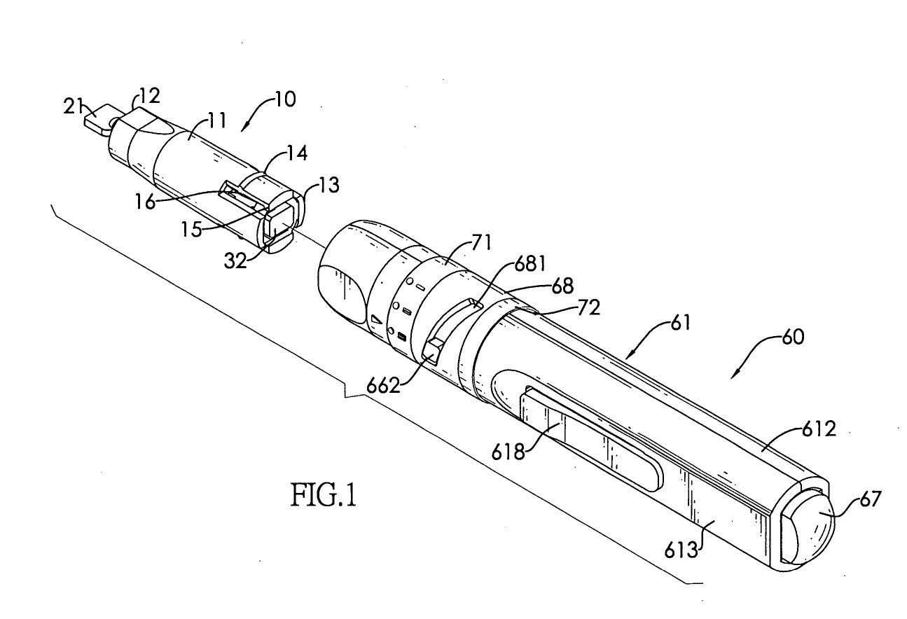

With reference to FIG. 1, a lancet device in accordance with the present invention comprises a needle assembly (10) and a lancet carrier (60).

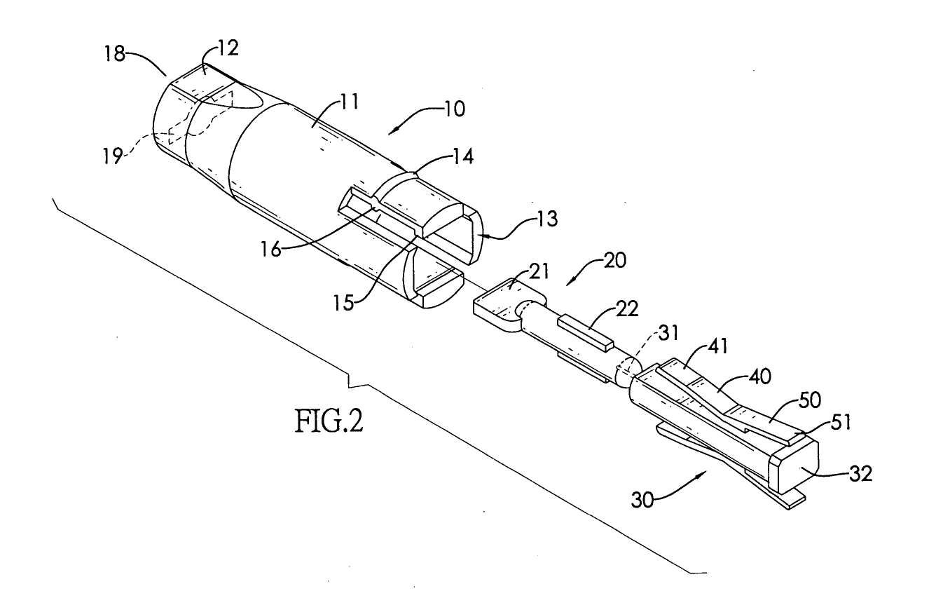

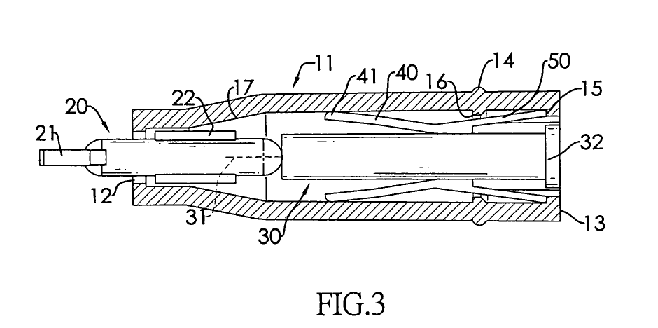

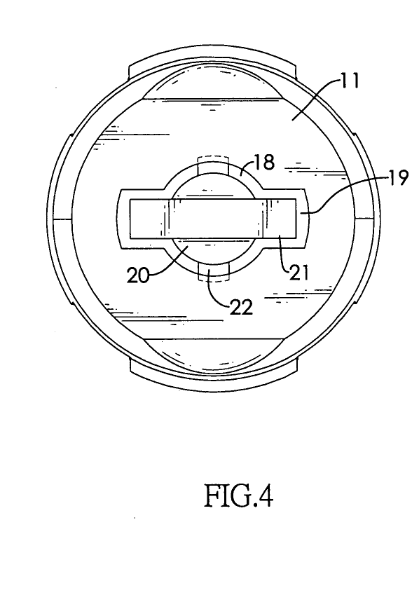

With further reference to FIGS. 2 to 4, the needle assembly (10) comprises a casing (11), a needle hub (30), a retracting unit (40), a limit unit (50) and an optional needle cap (20).

The casing (11) is cylindrical and hollow and comprises an axle, an outer surface, an inner surface, a front end (12), a rear end (13), a rear stop (15), a front stop (16), a neck (17), an opening (18), multiple optional circumference protrusions (14) and two optional lateral notches (19). The rear stop (15) is formed on the inner surface of the casing (10) near the rear end (13). The front stop (16) is formed on the inner surface of the casing (10) between the rear end (13) and the front end (12). The neck (17) is formed on the casing (10) near the front end (12). The opening (18) is formed through the front end (12) and is optionally circular. The circumference protrusions (14) are formed on and protrude from the outer surface of the casing (10) and are vertical to the axle of the casing (10). The lateral notches (19) are defined in the front end (12), communicate with and extend radially from the opening (18) and are opposite to each other.

The needle hub (30) is mounted in the casing (11) and comprises a top, a bottom, a front end, a rear end, a needle (31) and an optional flange (32). The needle (31) is mounted in and protrudes coaxially from the front end. The flange (32) is formed on and radially extends from the rear end.

The retracting unit (40) is formed on the top and the bottom of the needle hub (30). In a preferred embodiment, the retracting unit (40) comprises two resilient tabs formed respectively on the top and the bottom of the needle hub (30) and extending toward the front end of the needle hub (30). Each resilient tab comprises a proximal end and a distal end (41). The proximal end is connected the needle hub (30). The distal end (41) abuts with the inner surface of the casing (11) and corresponds to the neck (17).

The limit unit (50) is formed on the needle hub (30). In a preferred embodiment, the limit unit (50) comprises two resilient tabs formed respectively on the top and the bottom of the needle hub (30) and extending toward the rear end of the needle hub (30). Each resilient tab and comprises a proximal end and a distal end (51). The proximal end is connected the needle hub (30). The distal end (51) abuts with the rear stop (15) on the casing (11) when the needle hub (30) is at an original position. The distal ends (51) of the resilient tabs abut with the front stop (16) when the needle hub (30) is at an extended position.

The needle cap (20) is attached to and extends from the front end of the needle hub (30), covers the needle (211), is removed from the needle hub (30) through the front end (12) of the casing (11) before the lancet device is used and comprises a cylindrical body, two longitudinal keys (22) and a tab (21). The cylindrical body is attached to the front end of the needle hub (30), covers the needle (31) and has a front end, a rear end and an outer surface. The rear end is attached to the front end of the needle hub (30), covers the needle (31) and is detached from the needle hub (30) by twisting the cylindrical body. The longitudinal keys (22) are longitudinally formed on the outer surface of the cylindrical body of the needle cap (20) opposite to each other and respectively align with and extend through the lateral notches (19) in the front end (12) of the casing (10). The tab (21) is formed on and extends from the front end of the cylindrical body of the needle cap (22) and is adapted for a user twisting the cylindrical body to detach the needle cap (20) from the needle hub (20).

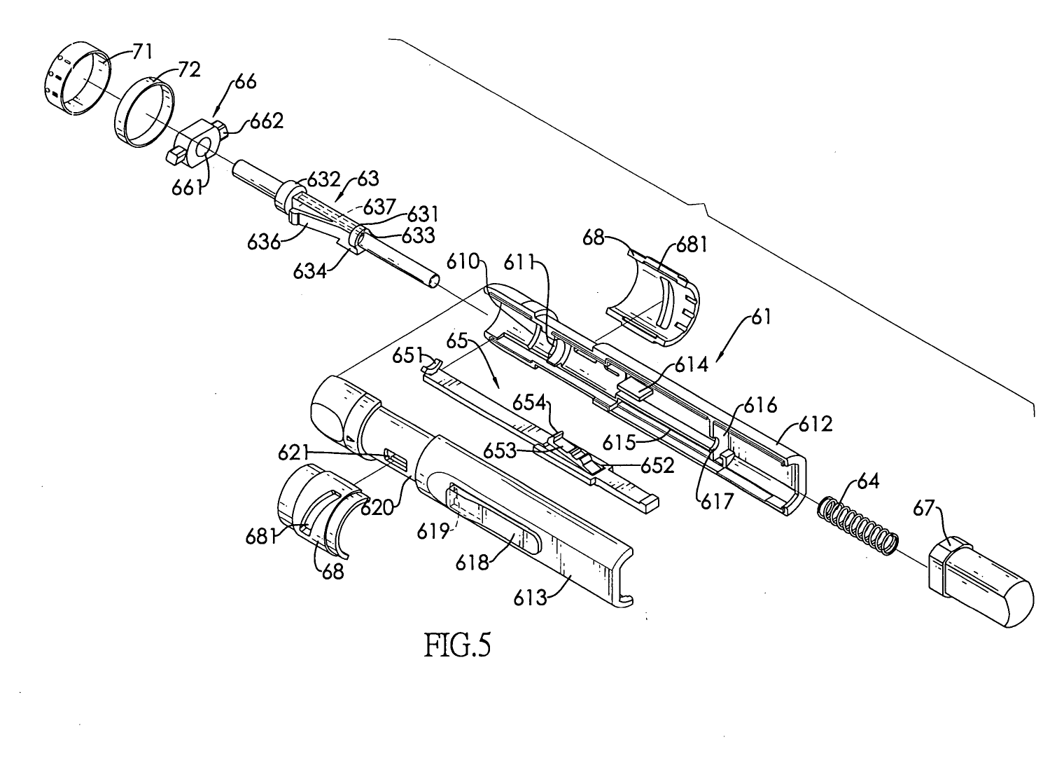

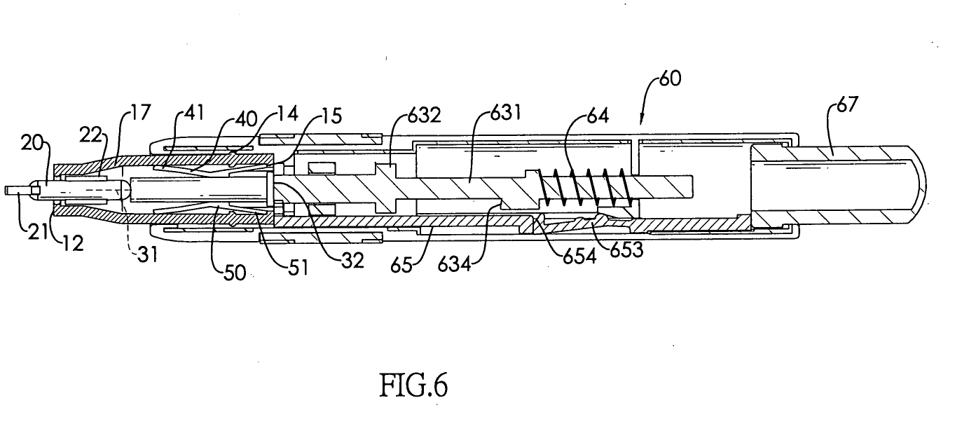

With reference to FIGS. 5 and 6, the lancet carrier (60) comprises a housing (61), a pushing rod (63), a spring (64), a pushing tab (65), an adjusting unit (66), a cap (67) and an optional collar (68).

The housing (61) comprises a front end, a rear end, an inner surface, an outer surface, a circular groove (610), an optional circular protrusion (611), a first half housing (612), a second half housing (613), a limit tab (614), a pressing unit (618), a protrusion (619), two adjusting holes (621) and an optional recess (620). The circular groove (610) is formed in the inner surface near the front end of the housing (61) to hold the circumference protrusion (14) of the needle assembly (10) inside. The circular protrusion (611) is formed on the inner surface of the housing (61) near the circular groove (610). The first half housing (612) comprises an inner surface and an outer surface. The second half housing (613) is combined with the first housing (612) to form the housing (61) and comprises an inner surface and an outer surface. The limit tab (614) is formed longitudinally on the inner surface of the first housing (612). Each one of the first half housing (612) and the second half housing (613) has a slot (615) formed in the inner surface and corresponding to each other. Each one of the first half housing (612) and the second half housing (613) has a stop tap (616) formed on the inner surface, corresponding to each other and vertical to the slot (615). Each stop tap (616) has a through hole (617) formed in the center of the stop tap (616) and corresponding to the through hole (617) in the other stop tap (616). The pressing unit (618) is mounted on the outer surface of the second half housing (613) and has a bottom. The protrusion (619) is formed on the inner surface of the second half housing (613) near the bottom of the pressing unit (618). The adjusting holes (621) are formed respectively through the outer surface of the first half housing (612) and the second half housing (613) and correspond to each other. The recess (620) is formed around the outer surface of the housing (61). The adjusting holes (621) are formed in the bottom of the recess (620).

The pushing rod (63) is mounted in the housing (61) and comprises a front end, a rear end, a middle (631), a front ring (632), a rear ring (633), a guide gap (637), a protrusion (634) and a resilient tab (636). The rear end of the pushing rod (63) extends through the through holes (617) in the stop taps (616). The middle (631) has two ends and the ends are formed respectively with the front ring (632) and the rear ring (633). The guide gap (637) is formed longitudinally in the middle (631), corresponds to the limit tab (614) on the first housing (612) and slidably holds the limit tab (614) inside. The protrusion (634) is formed on the rear ring (633). The resilient tab (636) is formed on the rear ring (633) and extends toward the front ring (532).

The spring (64) is mounted around the pushing rod (63) between the rear ring (633) of the pushing rod (63) and the stop tap (616).

The pushing tab (65) is mounted in housing (61) and comprises a front end, a rear end, a frame (652), a resilient tab (653), a pulling protrusion (654) and an optional curved tab (651). The front end of the pushing tab (65) is connected to the casing (11) of the needle assembly (10). Preferably, the front end is provided with the curved tab (651) to attach the casing (11) of the needle assembly (10). The curved tab (651) abuts with the circular protrusion (611) after the needle assembly (10) is inserted into the housing (61). The frame (652) is formed on the pushing tab (65) and is slidably held in the slots (615) in the first half housing (612) and the second half housing (613). The resilient tab (653) is formed on and extends from the frame (652) and has a free end. The pulling protrusion (654) is formed on the free end of the resilient tab (653) and attaches to the protrusion (634) on the pushing rod (63). Preferably, the pulling protrusion (654) has a top surface. The top surface inclines to the protrusion (634) of the pushing rod (63).

The adjusting unit (66) is mounted in the housing (61) and comprises a circular hole (661) and two bars (662). The circular hole (661) is formed through the adjusting unit (66) and is mounted around the pushing rod (63). The bars (662) are formed on the adjusting unit (66) opposite to each other and extend respectively through the adjusting holes (621).

The cap (67) is mounted on the rear end of the housing (61) and abuts with the pushing tab (65).

The collar (68) is mounted in the recess (620) in the housing (61) and comprises two slanted holes (681). The slanted holes (681) are formed in the collar (68) and correspond respectively to the adjusting holes (621), so the bars (662) of the adjusting unit (66) extend respectively into the slanted holes (681). Turning the collar (68) around the housing (61) pushes the bars (662) to move the adjusting unit (66) relative to the housing (61) along the adjusting holes (621). The collar (68) further comprises two rings (71,72). The rings (71,72) are mounted around the collar (68). One of the rings (71) has markers to indicate the location of the adjusting unit (66).

After inserting the needle assembly (10) into the lancet carrier (60), the rear end of the casing (11) contacts with the curved tab (651) of the pushing tab (65) to move the pushing tab (65) to abut with the cap (67). At the same time, the pulling protrusion (654) engages with the protrusion (634) of the pushing rod (63) to move the pushing rod (63) toward the cap (67) and compress the spring (64). The pushing rod (63) will slide along the limit tab (614) until the front end of the guide gap (637) abuts with the limit tab (614). When the pushing rod (63) is stopped by the limit tab (614), the pulling protrusion (654) will disengage from the protrusion (634) of the pushing rod (63) and keep moving toward the cap (67). The resilient tab (636) engages with the protrusion (619) on the pressing unit (618).

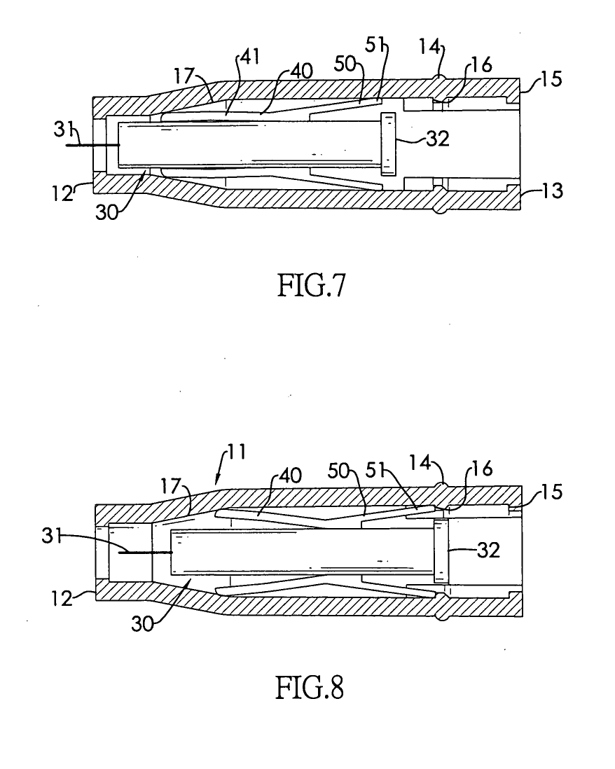

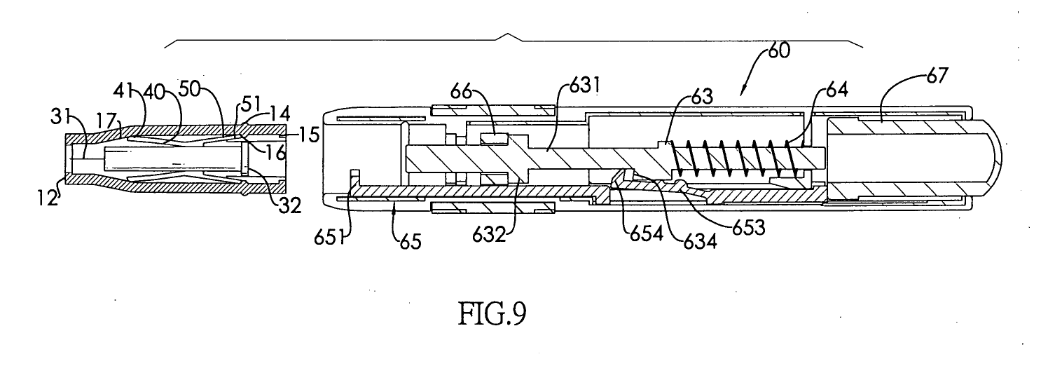

With reference to FIGS. 6 to 9, after turning the needle cap (20) to make the longitudinal keys (22) align respectively with the lateral notches (19), the needle cap (20) is removed from the needle hub (30) and is pulled out of the opening (18) to expose the needle (31) from the casing (11). When a user pushes the pressing unit (618), the resilient tab (636) will release from the protrusion (619) such that the pushing rod (63) is pushed to move into the needle assembly (10) by the resilient force provided by the spring (64). Then, the front end of the pushing rod (63) hits the flange (32) so the needle (31) is extended from the opening (18) to prick the skin of a person. The resilient tabs (41) of the retracting unit (40) contacts with and is compressed by the neck (17). After the force of the spring (64) is released, the force of the resilient tab (41) pushes the needle hub (30) to move backward and to make the needle (51) retract into the casing (11). Therefore, the needle (31) can not prick another person. At the same time, the resilient tabs (51) of the limit unit (50) engage with the front stop (16), such that the used needle assembly (10) can not be used again. After pressing the cap (67) to push the pushing tab (65), the pulling protrusion (654) moves to reengage with the protrusion (634) on the pushing rod (63). A new needle assembly (10) can be mounted in the lancet carrier to use again.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A needle assembly of lancet device comprising
a casing being cylindrical and hollow and comprising
an axle;
an outer surface;
an inner surface;
a front end;
a rear end;
a rear stop formed on the inner surface of the casing near the rear end;
a front stop formed on the inner surface of the casing between the rear end and the front end;
a neck formed near the front end; and
an opening defined in the front end;
a needle hub mounted in the casing and comprising
a top;
a bottom;
a front end;
a rear end; and
a needle mounted in and protruding coaxially from the front end;
a retracting unit formed on the top and the bottom of the needle hub; and
a limit unit formed on the needle hub.

2. The needle assembly of lancet device as claimed in claim 1, wherein
the retracting unit comprises two resilient tabs formed respectively on the top and the bottom of the needle hub and extending toward the front end of the needle hub; and
the limit unit comprises two resilient tabs formed respectively on the top and the bottom of the needle hub and extending toward the rear end of the needle hub.

3. The needle assembly of lancet device as claimed in claim 2, wherein the casing further comprises multiple circumference protrusions protruding on the outer surface of the casing and being vertical to the axle of the casing.

4. The needle assembly of lancet device as claimed in claim 3, wherein the casing further comprises two lateral notches defined in the front end, communicating with the opening and opposite to each other.

5. The needle assembly of lancet device as claimed in claim 4, wherein the needle assembly further comprises a needle cap comprising
a cylindrical body attached to the front end of the needle hub, covering the needle and having
a front end;
a rear end attached to the front end of the needle hub; and
an outer surface;
two longitudinal keys longitudinally formed on the outer surface of the body of the needle cap and opposite to each other; and
a tab formed on and extending from the front end of the cylindrical body of the needle cap.

6. A lancet device comprising
a needle assembly comprising
a casing being cylindrical and hollow and comprising
an axle;
an outer surface;
an inner surface;
a front end;
a rear end;
a rear stop formed on the inner surface of the casing near the rear end;
a front stop formed on the inner surface of the casing between the rear end and the front end;
a neck formed near the front end; and
an opening defined in the front end;
a needle hub mounted in the casing and comprising
a top;
a bottom;
a front end;
a rear end; and
a needle mounted in and protruding coaxially from the front end;
a retracting unit formed on the top and the bottom of the needle hub; and
a limit unit formed on the needle hub; and
a lancet carrier comprising
a housing comprising
a front end;
a rear end;
an inner surface;
an outer surface;
a circular groove formed in the inner surface near the front end of the housing to hold the protrusion of the needle assembly inside;
a circular protrusion formed on the inner surface of the housing;
a first half housing comprising
an inner surface;
an outer surface;
a slot formed in the inner surface of the first half housing; and
a stop tap formed on the inner surface of the first half housing, being vertical to the slot and having a through hole formed in a center of the stop tap;
a second half housing combined with the first housing to form the housing and comprising
an inner surface;
an outer surface;
a slot formed in the inner surface of the second half housing and corresponding to the slot in the first half housing; and
a stop tap formed on the inner surface of the second half housing, corresponding to the stop tap on the first half housing and having a through hole formed in a center of the stop tap and corresponding to the through hole in the stop tap on the first half housing;
a limit tab formed longitudinally on the inner surface of the first half housing;
a pressing unit mounted on and protruding from the outer surface of the second half housing and having a bottom;
a protrusion formed on the inner surface of the second half housing near the bottom of the pressing unit; and
two adjusting holes formed respectively through the outer surfaces of the first half housing and the second half housing;
a pushing rod slidably mounted in the housing and comprising
a front end;
a rear end extending through the through holes in the stop taps;
a front ring;
a rear ring;
a middle having
two ends formed respectively with the front ring and the rear ring;
a guide gaps formed longitudinally in the middle and corresponding to and holding the limit tab of the first half housing;
a protrusion formed on the rear ring; and
a resilient tab formed on the rear ring and extending toward the front ring;
a spring mounted around the pushing rod between the rear ring of the pushing rod and the stop taps;
a pushing tab slidably mounted in the slots in the first half housing and the second half housing and comprising
a front end contacting with the casing of the needle assembly;
a rear end;
a frame formed on the pushing tab and slidably held between the slots of the first half housing and the second half housing;
a resilient tab formed on the frame and having a free end; and
a pulling protrusion formed on the free end of the resilient tab and selectively engaging with the protrusion of the pushing rod;
an adjusting unit mounted in the housing and comprising
a circular hole formed through the adjusting unit and mounted around the pushing rod; and
two bars formed on the adjusting unit, opposite to each other and extending respectively out from the adjusting holes; and
a cap mounted on the rear end of the housing and abutting with the pushing tab.

7. The lancet device as claimed in claim 6, wherein
the pushing tab further comprises a curved tab formed on the front end;
the housing further comprises a circular protrusion formed on the inner surface of the housing; and
the curved tab abuts with the circular protrusion when the needle assembly inserted into the housing.

8. The lancet device as claimed in claim 7, wherein
the housing further comprises a recess formed around the outer surface of the housing and having a bottom, where the adjusting holes are defined in the bottom of the recess; and
the collar is mounted in the recess to move the adjusting unit while the collar is rotated.

9. The lancet device as claimed in claim 7, wherein the collar further comprises two rings being mounted around the collar and one of the rings has markers to indicate the location of the adjusting unit.
